(19) United States
(12) Patent Application Publication
(10) Pub. No.: US 2023/0088383 A1
(43) Pub. Date: Mar. 23, 2023

(54) METHOD FOR ASSESSING DIFFERENTIATION STATE OF CELLS, GELATIN NANOPARTICLES AND GELATIN NANOPARTICLE SET

(71) Applicant: Konica Minolta, Inc., Tokyo (JP)

(72) Inventors: TEMMEI ITO, Sagamihara-shi, Kanagawa (JP); AKIHIRO MAEZAWA, Hino-shi, Tokyo (JP); YASUHIKO TABATA, Uji-shi, Kyoto (JP); YUKI MURATA, Kyoto-shi, Kyoto (JP)

(21) Appl. No.: 17/910,206

(22) PCT Filed: Mar. 12, 2020
(86) PCT No.: PCT/JP2020/010702

§ 371 Date: Sep. 8, 2022

(51) Int. Cl. C12Q 1/6841; C12Q 1/6876

(52) U.S. Cl. CPC C12Q 1/6841; C12Q 1/6876; C12Q 2600/158

(57) ABSTRACT

An object of the present invention is to provide a method for assessing a differentiation state of cells, capable of assessing a differentiation state of a wide variety of cells, and gelatin nanoparticles and a gelatin nanoparticle set that can be used in the method. The purpose is achieved by a method for assessing a differentiation state of cells, the method including a step of observing expression of an mRNA encoding a peroxisome proliferator-activated receptor γ coactivator-1α (PGC-1α) or the peroxisome proliferator-activated receptor γ coactivator-1α (PGC-1α) in cells. The method can be performed by gelatin nanoparticles for assessing a differentiation state of cells, the gelatin nanoparticles carrying a probe capable of detecting an mRNA encoding PGC-1α or PGC-1α

### TECHNICAL FIELD

The present invention relates to a method for assessing a differentiation state of cells, gelatin nanoparticles, and a gelatin nanoparticle set.

### BACKGROUND ART

It is required to grasp a differentiation state of cells in various fields such as regenerative medicine, discovery of a disease, and treatment of a disease.

Conventionally, assessment of the differentiation state of cells has been performed by immunostaining or quantification of an expression level of a marker gene. However, by these methods, the differentiation state of cells cannot be assessed over time, and the differentiation state cannot be assessed for each cell.

Meanwhile, Patent Literature 1 describes a method for monitoring differentiation of cardiomyocytes over time by introducing a reporter gene of a luminescent protein configured to emit light according to expression of a myocardial differentiation marker gene into the cardiomyocytes. In Patent Literature 1, a vector incorporating a promoter of the marker gene and a gene of a luminescent protein (such as luciferase) located downstream thereof is introduced into cells by an electroporation method. When a transcription factor is synthesized and the myocardial differentiation marker gene is expressed, a luminescent protein derived from the vector is also expressed and emits light. Patent Literature 1 describes that the differentiation of cardiomyocytes can be monitored by observing this emission.

### CITATION LIST

### Patent Literature

Patent Literature 1: JP 2015-77122 A

### SUMMARY OF INVENTION

### Technical Problem

As described in Patent Literature 1, there is a need to develop a method capable of assessing differentiation of cells over time.

However, by a method using a differentiation marker gene specific to specific cells, such as the method described in Patent Literature 1, only a differentiation state of the specific cells can be assessed. Therefore, in order to assess a differentiation state of other cells, it is necessary to search for a differentiation marker gene of the other cells that can be used for assessing the differentiation state.

The present invention has been made based on the above findings, and an object of the present invention is to provide a method for assessing a differentiation state of cells, capable of assessing differentiation states of a wide variety of cells, and gelatin nanoparticles and a gelatin nanoparticle set that can be used in the method.

### Solution to Problem

The above problem is solved by a method for assessing a differentiation state of cells, the method including a step of detecting an mRNA encoding a peroxisome proliferator-activated receptor γ coactivator-1α

(PGC-1α) or the peroxisome proliferator-activated receptor γ coactivator-1α (PGC-1α) in cells.

In addition, the above problem is solved by gelatin nanoparticles for assessing a differentiation state of cells, the gelatin nanoparticles carrying a probe capable of detecting an mRNA encoding a peroxisome proliferator-activated receptor γ coactivator-1α (PGC-1α) or the peroxisome proliferator-activated receptor γ coactivator-1α (PGC-1α).

In addition, the above problem is solved by a gelatin nanoparticle set for assessing a differentiation state of cells, the set including: gelatin nanoparticles carrying a probe capable of detecting an mRNA encoding a peroxisome proliferator-activated receptor γ coactivator-1α (PGC-1α) or the peroxisome proliferator-activated receptor γ coactivator-1α (PGC-1α); and gelatin nanoparticles carrying a probe capable of detecting an mRNA (Pdk1) encoding pyruvate dehydrogenase kinase 1 or the pyruvate dehydrogenase kinase 1 (PDK1).

### Advantageous Effects of Invention

The present invention provides a method for assessing a differentiation state of cells, capable of assessing differentiation states of a wide variety of cells, and gelatin nanoparticles and a gelatin nanoparticle set that can be used in the method.

### BRIEF DESCRIPTION OF DRAWINGS

FIG. 1 is a flowchart illustrating a method for assessing a differentiation state of cells according to an embodiment of the present invention.

FIG. 2A is a graph illustrating an expression level of an mRNA of an undifferentiation marker in Test 1, and FIG. 2B is a graph illustrating an expression level of an mRNA of an initial differentiation marker in Test 1.

FIG. 3A is a graph illustrating an expression level of an mRNA encoding PGC-1α in a medium with addition of LIF (w LIF) or without addition of LIF (wo LIF) in Test 1, and FIG. 3B is a graph illustrating an expression level of pdk1 in a medium with addition of LIF (w LIF) or without addition of LIF (wo LIF) in Test 1.

FIG. 4A illustrates a fluorescence image (right side) of a medium one day after addition of cGNS (PGC-1α MB) under a condition with addition of LIF, and an image (left side) obtained by superimposing a bright field image and the fluorescence image in Test 1. FIG. 4B illustrates a fluorescence image (right side) of a medium two days after addition of cGNS (PGC-1α MB) under a condition with addition of LIF, and an image (left side) obtained by superimposing a bright field image and the fluorescence image in Test 1. FIG. 4C illustrates a fluorescence image (right side) of a medium three days after addition of cGNS (PGC-1α MB) under a condition with addition of LIF, and an image (left side) obtained by superimposing a bright field image and the fluorescence image in Test 1.

FIG. 5A illustrates a fluorescence image (right side) of a medium one day after addition of cGNS (PGC-1α MB) under a condition without addition of LIF, and an image (left side) obtained by superimposing a bright field image and the fluorescence image in Test 1. FIG. 5B illustrates a fluorescence image (right side) of a medium two days after addition of cGNS (PGC-1α MB) under a condition without addition of LIF, and an image (left side) obtained by superimposing a bright field image and the fluorescence image in Test 1. FIG. 5C illustrates a fluorescence image (right side) of a medium three days after addition of cGNS (PGC-1α MB) under a condition without addition of LIF, and an image (left side) obtained by superimposing a bright field image and the fluorescence image in Test 1.

FIG. 6A illustrates a fluorescence image (right side) of a medium one day after addition of cGNS (Pdk1 MB) under a condition with addition of LIF, and an image (left side) obtained by superimposing a bright field image and the fluorescence image in Test 1. FIG. 6B illustrates a fluorescence image (right side) of a medium two days after addition of cGNS (Pdk1 MB) under a condition with addition of LIF, and an image (left side) obtained by superimposing a bright field image and the fluorescence image. FIG. 6C illustrates a fluorescence image (right side) of a medium three days after addition of cGNS (Pdk1 MB) under a condition with addition of LIF, and an image (left side) obtained by superimposing a bright field image and the fluorescence image

FIG. 7A illustrates a fluorescence image (right side) of a medium one day after addition of cGNS (Pdk1 MB) under a condition without addition of LIF, and an image (left side) obtained by superimposing a bright field image and the fluorescence image in Test 1. FIG. 7B illustrates a fluorescence image (right side) of a medium two days after addition of cGNS (Pdk1 MB) under a condition without addition of LIF, and an image (left side) obtained by superimposing a bright field image and the fluorescence image in Test 1. FIG. 7C illustrates a fluorescence image (right side) of a medium three days after addition of cGNS (Pdk1 MB) under a condition without addition of LIF, and an image (left side) obtained by superimposing a bright field image and the fluorescence image in Test 1.

FIG. 8A illustrates a fluorescence image (right side) of a medium one day after addition of cGNS (Actb MB) under a condition with addition of LIF, and an image (left side) obtained by superimposing a bright field image and the fluorescence image in Test 1. FIG. 8B illustrates a fluorescence image (right side) of a medium two days after addition of cGNS (Actb MB) under a condition with addition of LIF, and an image (left side) obtained by superimposing a bright field image and the fluorescence image in Test 1. FIG. 8C illustrates a fluorescence image (right side) of a medium three days after addition of cGNS (Actb MB) under a condition with addition of LIF, and an image (left side) obtained by superimposing a bright field image and the fluorescence image in Test 1.

FIG. 9A illustrates a fluorescence image (right side) of a medium one day after addition of cGNS (Actb MB) under a condition without addition of LIF, and an image (left side) obtained by superimposing a bright field image and the fluorescence image in Test 1. FIG. 9B illustrates a fluorescence image (right side) of a medium two days after addition of cGNS (Actb MB) under a condition without addition of LIF, and an image (left side) obtained by superimposing a bright field image and the fluorescence image in Test 1. FIG. 9C illustrates a fluorescence image (right side) of a medium three days after addition of cGNS (Actb MB) under a condition without addition of LIF, and an image (left side) obtained by superimposing a bright field image and the fluorescence image in Test 1.

FIG. 10A is a graph illustrating a fluorescence intensity of a medium to which cGNS (PGC-1α MB) is added in Test 1, FIG. 10B is a graph illustrating a fluorescence intensity of a medium to which cGNS (Pdk1 MB) is added in Test 1, and FIG. 10C is a graph illustrating a fluorescence intensity of a medium to which cGNS (Actb MB) is added in Test 1.

FIG. 11A illustrates a fluorescence image (right side) of a medium to which cGNS (Pdk1 MB) is added, and an image (left side) obtained by superimposing a bright field image and the fluorescence image in Test 1. FIG. 11B illustrates a fluorescence image (right side) of a medium to which a complex of Lipofectamine 2000 and Pdk1 MB is added, and an image (left side) obtained by superimposing a bright field image and the fluorescence image in Test 1. FIG. 11C illustrates a fluorescence image (right side) of a medium to which Pdk1 MB alone is added, and an image (left side) obtained by superimposing a bright field image and the fluorescence image in Test 1.

FIG. 12A illustrates a fluorescence image (right side) of a medium to which cGNS (Actb MB) is added, and an image (left side) obtained by superimposing a bright field image and the fluorescence image in Test 1. FIG. 12B illustrates a fluorescence image (right side) of a medium to which a complex of Lipofectamine 2000 and Actb MB is added, and an image (left side) obtained by superimposing a bright field image and the fluorescence image in Test 1. FIG. 12C illustrates a fluorescence image (right side) of a medium to which Actb MB alone is added, and an image (left side) obtained by superimposing a bright field image and the fluorescence image in Test 1.

FIG. 13A is a graph illustrating an expression level of an mRNA encoding PGC-1α in Test 2, FIG. 13B is a graph illustrating an expression level of the mRNA of Pdk1 in Test 2, FIG. 13C is a graph illustrating an expression level of the mRNA of Oct-¾ in Test 2, and FIG. 13D is a graph illustrating an expression level of the mRNA of Sox2 in Test 2.

FIG. 14A is a graph illustrating an expression level of the mRNA of Nanog in Test 2, FIG. 14B is a graph illustrating an expression level of the mRNA of Pax6 in Test 2, FIG. 14C is a graph illustrating an expression level of the mRNA of Nestin in Test 2, and FIG. 14D is a graph illustrating an expression level of the mRNA of Tubb III in Test 2.

FIG. 15A illustrates a fluorescence image (right side) of a medium to which cGNS (PGC-1α MB) is added under a condition with addition of LIF, and an image (left side) obtained by superimposing a bright field image and the fluorescence image in Test 2. FIG. 15B illustrates a fluorescence image (right side) of a medium four days after addition of cGNS (PGC-1α MB) under a condition without addition of LIF, and an image (left side) obtained by superimposing a bright field image and the fluorescence image in Test 2. FIG. 15C illustrates a fluorescence image (right side) of a medium seven days after addition of cGNS (PGC-1α MB) under a condition without addition of LIF, and an image (left side) obtained by superimposing a bright field image and the fluorescence image in Test 2. FIG. 15D illustrates a fluorescence image (right side) of a medium nine days after addition of cGNS (PGC-1α MB) under a condition without addition of LIF, and an image (left side) obtained by superimposing a bright field image and the fluorescence image in Test 2.

FIG. 16A illustrates a fluorescence image (right side) of a medium to which cGNS (Pdk1 MB) is added under a condition with addition of LIF, and an image (left side) obtained by superimposing a bright field image and the fluorescence image in Test 2. FIG. 16B illustrates a fluorescence image (right side) of a medium four days after addition of cGNS (Pdk1 MB) under a condition without addition of LIF, and an image (left side) obtained by superimposing a bright field image and the fluorescence image in Test 2. FIG. 16C illustrates a fluorescence image (right side) of a medium seven days after addition of cGNS (Pdk1 MB) under a condition without addition of LIF, and an image (left side) obtained by superimposing a bright field image and the fluorescence image in Test 2. FIG. 16D illustrates a fluorescence image (right side) of a medium nine days after addition of cGNS (Pdk1 MB) under a condition without addition of LIF, and an image (left side) obtained by superimposing a bright field image and the fluorescence image in Test 2.

FIG. 17A illustrates a fluorescence image (right side) of a medium to which cGNS (Actb MB) is added under a condition with addition of LIF, and an image (left side) obtained by superimposing a bright field image and the fluorescence image in Test 2. FIG. 17B illustrates a fluorescence image (right side) of a medium four days after addition of cGNS (Actb MB) under a condition without addition of LIF, and an image (left side) obtained by superimposing a bright field image and the fluorescence image in Test 2. FIG. 17C illustrates a fluorescence image (right side) of a medium seven days after addition of cGNS (Actb MB) under a condition without addition of LIF, and an image (left side) obtained by superimposing a bright field image and the fluorescence image in Test 2. FIG. 17D illustrates a fluorescence image (right side) of a medium nine days after addition of cGNS (Actb MB) under a condition without addition of LIF, and an image (left side) obtained by superimposing a bright field image and the fluorescence image in Test 2.

FIG. 18A is a graph illustrating a fluorescence intensity of a medium to which cGNS (PGC-1α MB) is added in Test 2, FIG. 18B is a graph illustrating a fluorescence intensity of a medium to which cGNS (Pdk1 MB) is added in Test 2, and FIG. 18C is a graph illustrating a fluorescence intensity of a medium to which cGNS (Actb MB) is added in Test 2.

### DESCRIPTION OF EMBODIMENTS

Hereinafter, an embodiment of the present invention will be described in detail with reference to the drawings. Note that the present invention is not limited to the following embodiment.

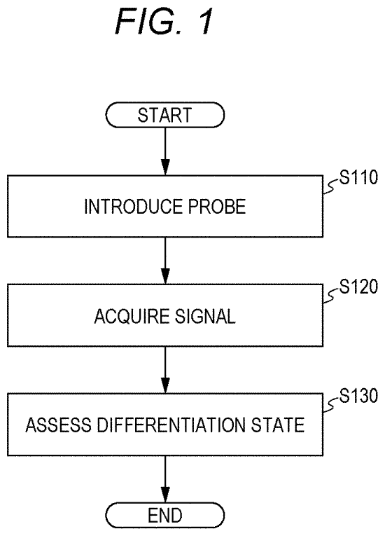

FIG. 1 is a flowchart illustrating a method for assessing a differentiation state of cells according to an embodiment of the present invention.

In the present embodiment, a probe is introduced into cells (step S110), a signal from the probe is acquired (step S120), and a differentiation state of the cells is assessed based on the acquired signal (step S130).

(Introduction of Probe (step S110))

First, a probe is introduced into cells.

The probe only needs to be able to detect an mRNA encoding a peroxisome proliferator-activated receptor γ coactivator-1α (PGC-1α) or the peroxisome proliferator-activated receptor γ coactivator-1α (PGC-1a). In the present embodiment, a metabolic state of cells is detected by detecting expression of the enzyme or the mRNA, and a differentiation state of the cells is thereby assessed.

The present inventors have found that an expression level of an mRNA encoding PGC-1α or PGC-1α is remarkably increased in differentiated somatic cells as compared with undifferentiated cells. In addition, the present inventors have found that detection of the expression level of the mRNA encoding PGC-1α or PGC-1α is extremely useful for determining a differentiation state of cells from an undifferentiation state in which metabolism by a glycolytic system is dominant to a differentiated state in which metabolism in mitochondria is activated, thereby completing the present invention.

Based on the above new findings, in this step, in order to detect the expression level of the mRNA encoding PGC-1α or PGC-1α, a probe capable of detecting the mRNA encoding PGC-1α or a probe capable of detecting PGC-1α is introduced into cells.

The probe only needs to be a compound having a site that directly or indirectly binds to the mRNA encoding PGC-1α or PGC-1α, and a site that emits a detectable signal. For example, the probe may be a probe capable of specifically binding to the mRNA encoding PGC-1α by a nucleic acid having a sequence complementary to at least a part of a nucleic acid sequence of the mRNA encoding PGC-1α, or may be a probe capable of specifically binding to PGC-1α by an antibody. In addition, the probe may be a probe that contains a phosphor and emits fluorescence as a signal, or may be a probe that emits another signal by chemiluminescence or the like.

The type of the phosphor is not particularly limited, and may be a fluorescent dye or a semiconductor nanoparticle.

Examples of the fluorescent dye include a rhodamine-based dye molecule, a squarylium-based dye molecule, a fluorescein-based dye molecule, a coumarin-based dye molecule, an acridine-based dye molecule, a pyrene-based dye molecule, an erythrosin-based dye molecule, an eosin-based dye molecule, a cyanine-based dye molecule, an aromatic ring-based dye molecule, an oxazine-based dye molecule, a carbopyronine-based dye molecule, and a pyrromethene-based dye molecule.

Examples of a semiconductor constituting the semiconductor nanoparticle include a group II-VI compound semiconductor, a group III-V compound semiconductor, and a group IV semiconductor. Specific examples of the semiconductor constituting the semiconductor nanoparticle include CdSe, CdS, CdTe, ZnSe, ZnS, ZnTe, InP, InN, InAs, InGaP, GaP, GaAs, Si, and Ge.

The probe capable of specifically binding to the mRNA encoding PGC-1α may be a known probe such as a molecular beacon, a Taqman probe, a cycling probe, or an INAF probe, but the molecular beacon is preferable because a general-purpose fluorescent dye can be used and detection for various types of cells is easy.

The molecular beacon is a nucleic acid derivative having a stem-loop structure, in which a fluorescent dye binds to one of a 5′ end and a 3′ end, and a quenching dye binds to the other end. In a state where the molecular beacon forms the stem-loop structure, the fluorescent dye and the quenching dye are close to each other, and therefore fluorescence emitted from the fluorescent dye is quenched. However, when the molecular beacon is close to a target sequence (mRNA encoding PGC-1α), the molecular beacon opens a loop structure and binds to the mRNA encoding PGC-1α. As a result, the fluorescent dye and the quenching dye are separated from each other, and fluorescence emission is detected.

A combination of the fluorescent dye and the quenching dye is not particularly limited, and only needs to be appropriately selected from the fluorescent dyes described above. The quenching dye may be a molecule that performs quenching by any of fluorescence resonance energy transfer (FRET), contact quenching, and collisional quenching.

The molecular beacon only needs to have a sequence complementary to at least a part of the nucleic acid sequence of the mRNA encoding PGC-1α. Note that the complementary sequence only needs to be sufficiently complementary to such an extent that the molecular beacon can bind to the mRNA encoding PGC-1α. For example, the complementary sequence only needs to have 80% or more identity to at least a part of the nucleic acid sequence of the mRNA encoding PGC-1α, preferably has 90% or more identity thereto, and more preferably has 95% or more identity thereto. The sequence complementary to at least a part of the nucleic acid sequence of the mRNA encoding PGC-1α typically constitutes the loop structure of the molecular beacon, and only needs to be, for example, a sequence including 2 or more and 40 or less nucleic acids.

In addition, the molecular beacon has mutually complementary sequences on both a 5′ end side and a 3′ end side of the sequence complementary to at least a part of the nucleic acid sequence of PGC-1α. The mutually complementary sequences bind to each other to constitute a stem region of the stem-loop structure. Each of the mutually complementary sequences only needs to be a sequence including, for example, 5 or more and 10 or less nucleic acids. In each of the mutually complementary sequences, the total amount of cytosine (C) and thymine (T) with respect to the total amount of adenine (A), cytosine (C), thymine (T), and guanine (G) is preferably 50% or more from a viewpoint of enhancing stability of the molecular beacon.

The probe capable of specifically binding to PGC-1α by the antibody is preferably a phosphor integrated dot (PID). The PID is a nano-sized particle whose base material is an organic or inorganic particle and which contains a plurality of phosphors. The PID binds directly or indirectly to an antibody that specifically binds to PGC-1α and labels PGC-1α. The plurality of phosphors may be present in the particle or on a surface of the particle. The phosphor integrated dot can emit fluorescence having an intensity sufficient to indicate each molecule of a target substance as a bright spot.

Examples of the organic substance to be the base material include: a thermosetting resin such as a melamine resin, a urea resin, an aniline resin, a guanamine resin, a phenol resin, a xylene resin, or a furan resin; a thermoplastic resin such as a styrene resin, an acrylic resin, an acrylonitrile resin, an acrylonitrile-styrene copolymer (AS resin), or an acrylonitrile-styrene-methyl acrylate copolymer (ASA resin); other resins such as polylactic acid; and a polysaccharide. Examples of the inorganic substance to be the base material include silica and glass.

Preferably, the base material and the fluorescent substance have substituents or sites having charges opposite to each other, and have electrostatic interaction to each other.

The average particle size of the phosphor integrated dots is not particularly limited, but is preferably 10 nm or more and 500 nm or less, and more preferably 50 nm or more and 200 nm or less in consideration of ease of detection as a bright spot and the like.

Note that the particle size of the phosphor integrated dot can be measured by measuring a projected area of the phosphor integrated dot using a scanning electron microscope (SEM) and converting the projected area into an equivalent circle diameter. The average particle size and the coefficient of variation of an aggregate including the plurality of phosphor integrated dots are calculated using a particle size (equivalent circle diameter) calculated for a sufficient number (for example, 1000) of phosphor integrated dots.

A method for introducing the probe into cells is not particularly limited, but in the present embodiment, the probe is preferably introduced into cells by gelatin nanoparticles carrying the probe.

The gelatin nanoparticles are taken into cells by the cells' own activity. Therefore, the gelatin nanoparticles make it possible to easily introduce the probe into cells while reducing an influence on activity of living cells as compared with other methods such as an electroporation method. In addition, the gelatin particles can carry a large amount of the probe, and therefore make it possible to introduce a large amount of the probe into cells at a time. Furthermore, the gelatin nanoparticles sustainably release the probe for a long time after the gelatin nanoparticles are taken into cells, and therefore make it possible to detect expression of the mRNA encoding PGC-1α or PGC-1α over time.

Furthermore, the probe capable of specifically binding to the mRNA encoding PGC-1α is formed by a negatively charged nucleic acid, and therefore hardly enters the inside of a negatively charged cell membrane as it is. Meanwhile, by causing the gelatin nanoparticles to carry the probe and causing the gelatin nanoparticles to be taken into cells, the probe can be more easily introduced into the cells.

The gelatin nanoparticles may be nanoparticles made of any known gelatin obtained by modifying collagen derived from bovine bone, cow skin, pig skin, pig tendon, fish scales, fish meat, and the like. Gelatin has been used for food and medical purposes for a long time, and is less harmful to a human body even when being ingested into the body. In addition, since gelatin is dispersed and disappears in a living body, there is an advantage that it is not necessary to remove gelatin from the living body.

The weight average molecular weight of gelatin constituting the gelatin nanoparticles is preferably 1000 or more and 100,000 or less. The weight average molecular weight can be, for example, a value measured in accordance with PAGI Method, Tenth Edition (2006).

Gelatin constituting the gelatin nanoparticles may be crosslinked. The crosslinking may be crosslinking with a crosslinking agent, or may be self-crosslinking performed without using a crosslinking agent.

The gelatin nanoparticles are preferably cationized by, for example, introducing a primary amino group, a secondary amino group, a tertiary amino group, or a quaternary ammonium group from a viewpoint of easily carrying the probe capable of detecting PGC-1α. A nucleic acid has a negative charge, and therefore can electrostatically interact with cationized gelatin to bind thereto more strongly.

Cationization of the gelatin nanoparticles can be performed by a known method for introducing a functional group to be cationized under physiological conditions at the time of production. For example, an alkyldiamine including ethylenediamine and N,N-dimethyl-1,3-diaminopropane, trimethylammonium acetohydrazide, spermine, spermidine, or diethylamide chloride can be reacted with a condensing agent including a dianhydride compound such as 1-ethyl-3-(3-dimethylaminopropyl) carbodiimide hydrochloride, cyanuric chloride, N,N′-carbodiimidazole, cyanogen bromide, a diepoxy compound, tosyl chloride, or diethyltriamine-N,N,N′,N″,N″-pentanoic acid dianhydride, and tricyl chloride to introduce the amino group into a hydroxy group or a carboxyl group of gelatin.

The gelatin nanoparticles carry the probe. For example, when the probe is a molecular beacon, the gelatin nanoparticles carry the molecular beacon. When the probe is PID, the gelatin nanoparticles carry PID, an antibody that specifically binds to PGC-1α, and a medium molecule that binds the antibody to PID.

The gelatin nanoparticles carrying the probe means that the probe is immobilized on surfaces of the gelatin nanoparticles or taken into the gelatin nanoparticles.

Note that the amount of the probe in the gelatin nanoparticles is preferably larger than the amount of the probe in a surface layer portion. By reducing the amount of the probe in the surface layer portion of the gelatin nanoparticles, the amount of the probe exposed on the surfaces of the gelatin nanoparticles can be reduced. This makes it difficult for cells to recognize the gelatin nanoparticles as a foreign substance, and makes it easy for the gelatin nanoparticles to be taken into the cells by an activity such as endocytosis. The surface layer portion means a region up to a depth of 1% with respect to the average particle size of the gelatin nanoparticles.

The average particle size of the gelatin nanoparticles is preferably 100 nm or more and 1000 nm or less. Although the gelatin nanoparticles carry the probe, the gelatin nanoparticles do not substantially have the probe in the surface layer portion thereof. Therefore, even when the average particle size is 1000 nm, the gelatin nanoparticles are easily taken into cells by the cells' own activity. In order to cause many gelatin nanoparticles to be taken into cells in a shorter time, the average particle size of the gelatin nanoparticles is more preferably 800 nm or less. On the other hand, the gelatin nanoparticles having an average particle size of 100 nm or more easily carry the probe in the particles, and can increase the amount of the probe to be housed therein. The average particle size of the gelatin nanoparticles is preferably 200 nm or more, and more preferably 300 nm or more from the above viewpoint.

Note that the average particle size of the gelatin nanoparticles can be an apparent particle size of each of the gelatin nanoparticles measured by a dynamic light scattering method. Alternatively, the average particle size of the gelatin nanoparticles can be a value obtained by addition-averaging a major axis and a minor axis. The minor axis and major axis of each of the gelatin nanoparticles can be values obtained by analyzing an image obtained by imaging dried gelatin nanoparticles after being left to stand in the air at 80° C. for 24 hours with a scanning electron microscope (SEM). Since the gelatin nanoparticle is usually in a form of an aggregate formed of a plurality of gelatin nanoparticles, the major axis, minor axis, and particle size of the gelatin nanoparticle can be values obtained by addition-averaging the major axis, minor axis, and particle size of a plurality of gelatin nanoparticles (for example, 20 gelatin nanoparticles) arbitrarily selected from the aggregate, respectively. When there is a difference between average particle sizes measured by these methods, it is only required to adopt an average particle size obtained by measurement by a dynamic light scattering method.

The amount of the probe carried by the gelatin nanoparticles, an average concentration of the probe in a surface layer portion of the gelatin nanoparticles, and an average concentration of the probe in the gelatin nanoparticles can be determined by XPS depth profile measurement. In the XPS depth profile measurement, by concurrently using measurement of X-ray photoelectron spectroscopy (XPS) and ion sputtering of a rare gas such as argon, it is possible to sequentially perform surface composition analysis while exposing the inside of a sample. A distribution curve obtained by such measurement can be created, for example, with the vertical axis as an atomic ratio (unit: at %) of each element and the horizontal axis as etching time (sputtering time). Note that, in the distribution curve of an element with the horizontal axis as etching time, the etching time is roughly correlated with a distance from a surface. Therefore, elemental analysis from the surface of the gelatin nanoparticle to the center thereof is performed, and a distribution curve of an element of the gelatin nanoparticles is determined. The amount of the probe in the surface layer portion can be determined from an elemental distribution from a measurement start point to an etching time corresponding to 0.01X (X is the average particle size), and the amount of the probe in the gelatin nanoparticle can be determined from an elemental distribution from the etching time corresponding to 0.01X to an etching time corresponding to the center of the particle.

The amount of the probe is measured at a plurality of arbitrarily selected locations (for example, ten locations) by the above method, an average value (mass) of the probe contained in each of the surface layer portion and the inside is determined, a concentration thereof with respect to the total mass of the gelatin particles (that is, the total mass of the gelatin and the probe) is determined and can be adopted as an average concentration of each of these. Since the gelatin nanoparticle is usually in a form of an aggregate formed of a plurality of particles, the average concentration of the probe can be a value obtained by addition-averaging an average concentration of a plurality of gelatin particles (for example, 20 gelatin particles) arbitrarily selected from the aggregate.

The gelatin nanoparticle carrying the probe is brought into contact with cells to be taken into the cells by the cells' own activity.

The cells only need to be cells whose differentiation state is to be assessed, are preferably cells in which a state in which metabolism by a glycolytic system is dominant and a state in which metabolism in mitochondria is activated are switched by differentiation or dedifferentiation, and particularly preferably cells in which a glycolytic system is dominant in an undifferentiation state and metabolism in mitochondria is activated in a differentiated state. Examples of the cells include stem cells including embryonic stem cells (ES cells) and induced pluripotent stem cells (iPS cells), nerve cells, and cancer cells.

For example, when cells or tissues induced to be differentiated from pluripotent stem cells are transplanted into a living body, if undifferentiated pluripotent stem cells remain, tumor may be formed. Therefore, it is expected that safety of regenerative medicine such as transplantation is improved by introducing the probe into pluripotent stem cells and assessing a differentiation state of the cells.

Note that the cells may be differentiated somatic cells derived from a biological sample or a specimen extracted from various organs, instead of undifferentiated cells. By introducing the probe into these cells and observing whether or not expression of the mRNA encoding PGC-1α or PGC-1α decreases, it is also possible to assess canceration of these cells or acquisition of pluripotency due to dedifferentiation

These cells are collected from a living body, and the probe is introduced into the cells by a known method. The introduction may be performed by a known method such as an electroporation method or a microinjection method, but a method for mixing and culturing the gelatin nanoparticles carrying the probe and cells in a liquid is preferable from a viewpoint of suppressing a decrease in activity of the cells.

In this step, a probe capable of detecting an mRNA encoding pyruvate dehydrogenase kinase 1 (Pdk1) or pyruvate dehydrogenase kinase 1 (PDK1) may be introduced.

Metabolism of cells includes a glycolytic system, which takes place in the cytoplasm, and a TCA cycle and oxidative phosphorylation, which take place in mitochondria. It is known that metabolism by a glycolytic system is dominant in undifferentiated cells, but metabolism in mitochondria (TCA cycle and oxidative phosphorylation) is also activated in differentiated somatic cells.

Pyruvic acid, which is a final product of a glycolytic system, is oxidatively decarboxylated by a complex containing pyruvate dehydrogenase (PDH), dihydrolipoamide transacetylase, and dihydrolipoamide dehydrogenase (pyruvate dehydrogenase complex (PDC)), converted to acetyl CoA, and sent to a TCA cycle. Then, PDH is phosphorylated by four PDH kinases, PDK1, PDK2, PDK3, and PDK4, and activity thereof is inhibited. PDH is dephosphorylated by two PDH phosphatases, pyruvate dehydrogenase phosphatase 1 (PDP1) and PDP2, and activity is imparted thereto.

The above many enzymes and coenzymes thereof are involved in the conversion of pyruvic acid to acetyl CoA. However, it has been unknown whether or not expression of these enzymes or coenzymes is useful as a marker of differentiation of cells, and it has been unknown which enzyme or coenzyme serves as a marker if these enzymes or coenzymes can serve as a marker.

Meanwhile, the present inventors have found that an expression level of pdk1 or PDK1 is remarkably increased in undifferentiated cells as compared with differentiated somatic cells. In addition, the present inventors have found that detection of the expression level of pdk1 or PDK1 is extremely useful for determining a differentiation state of cells from an undifferentiation state in which metabolism by a glycolytic system is dominant to a differentiated state in which metabolism in mitochondria is activated.

The probe only needs to be a compound having a site that directly or indirectly binds to pdk1 or PDK1, and a site that emits a detectable signal. For example, the probe may be a probe capable of specifically binding to pdk1 by a nucleic acid having a sequence complementary to at least a part of a nucleic acid sequence of pdk1, or may be a probe capable of specifically binding to PDK1-1α by an antibody. In addition, the probe may be a probe that contains a phosphor and emits fluorescence as a signal, or may be a probe that emits another signal by chemiluminescence or the like.

The configurations of these probes can be similar to those of the probe capable of detecting the mRNA encoding PGC-1α and the probe capable of detecting PGC-1α.

At this time, the gelatin nanoparticles may be a gelatin nanoparticle set containing gelatin nanoparticles carrying the probe capable of detecting the mRNA encoding PGC-1α or PGC-1α and gelatin nanoparticles carrying the probe capable of detecting Pdk1 or PDK1.

At this time, as a control, a probe capable of detecting an mRNA (for example, Actb) or a protein (for example, β actin (ACTB)) whose expression level does not change depending on a differentiation state of cells may be introduced. The configuration of this probe can also be similar to that of the probe capable of detecting the mRNA encoding PGC-1α and the probe capable of detecting PGC-1α.

At this time, the gelatin nanoparticles may be a gelatin nanoparticle set containing gelatin nanoparticles carrying the probe capable of detecting the mRNA encoding PGC-1α or PGC-1α, gelatin nanoparticles carrying the probe capable of detecting Pdk1 or PDK1, and gelatin nanoparticles carrying the probe capable of detecting an mRNA or a protein whose expression level does not change depending on a differentiation state of cells.

(Acquisition of Signal From Probe (step S120))

Next, a signal derived from the probe, the signal being emitted from cells into which the probe has been introduced, is acquired. As a result, expression of PDK1 or Pdk1 in the cells can be detected.

The signal may be acquired by a method according to the type of a signal emitted from the probe. For example, when the probe contains a phosphor, it is only required to image fluorescence emitted from the cells using a fluorescence microscope or the like to obtain a fluorescence image.

The signal may be acquired by a method capable of confirming presence or absence of the signal or by a method for quantitatively measuring the signal amount of the signal. The signal may be acquired by a qualitative method or a quantitative method.

The signal may be acquired immediately after the probe is introduced, or after a predetermined time has elapsed. The signal may be acquired only once or over time (consecutively or a plurality of times at intervals). When it is desired to determine the current state of the cells, it is only required to acquire the signal immediately after the probe is introduced. When it is desired to observe a timing at which the cells are differentiated, it is only required to acquire the signal over time after the probe is introduced.

In particular, when the probe is introduced into cells by gelatin nanoparticles carrying the probe, the gelatin nanoparticles sustainably release the probe, and it is thereby easy to acquire the signal over time.

The cells are maintained in a viable state until the signal is acquired. At this time, the cells may be cultured in a medium or may be returned to a living body. At this time, differentiation or dedifferentiation of the cells may be promoted, or differentiation or dedifferentiation may be inhibited.

(Assessment of Differentiation State of Cells (step S130))

Based on the obtained signal, a differentiation state of the cells can be assessed.

According to new findings by the present inventors, an expression level of PGC-1α changes with differentiation of cells. When the cells are undifferentiated and metabolism by a glycolytic system is dominant, an expression level of the mRNA encoding PGC-1α or PGC-1α is lower. Conversely, when cells are differentiated and metabolism in mitochondria is activated, the expression level of the mRNA encoding PGC-1α or PGC-1α is higher. Therefore, when the expression level of the mRNA encoding PGC-1α or PGC-1α is lower, it can be determined that the cells are undifferentiated, and when the expression level of the mRNA encoding PGC-1α or PGC-1α is higher, it can be determined that the cells are differentiated. By observing the cells into which the probe has been introduced over time, when the expression level of the mRNA encoding PGC-1α or PGC-1α increases, it can be determined that the cells are differentiated, and when the expression level of the mRNA encoding PGC-1α or PGC-1α decreases, it can be determined that the cells are dedifferentiated.

According to new findings of the present inventors, an expression level of PDK1 also changes with differentiation of cells. When cells are undifferentiated and metabolism by a glycolytic system is dominant, the expression level of Pdk1 or PDK1 is higher. Conversely, when cells are differentiated and metabolism in mitochondria is activated, the expression level of Pdk1 or PDK1 is lower. Therefore, when the expression level of Pdk1 or PDK1 is higher, it can be determined that the cells are undifferentiated, and when the expression level of Pdk1 or PDK1 is lower, it can be determined that the cells are differentiated. By observing the cells into which the probe has been introduced over time, when the expression level of Pdk1 or PDK1 decreases, it can be determined that the cells are differentiated, and when the expression level of Pdk1 or PDK1 increases, it can be determined that the cells are dedifferentiated.

By observing the expression levels of the mRNAs encoding these two types of proteins or the proteins over time, the differentiation state of cells can also be observed in a superimposed manner.

### EXAMPLES

Hereinafter, specific Examples of the present invention will be described together with Comparative Examples, but the present invention is not limited thereto.

Note that, in the drawings related to the following description, “*” indicates that there is a significant difference (a p value of less than 0.05 is regarded as being statistically significant), and “ns” indicates that there is no significant difference.

The following experiment was performed using a molecular beacon capable of detecting the mRNA encoding PGC-1α, a molecular beacon capable of detecting Pdk1, and a molecular beacon capable of detecting an mRNA of β-actin (Actb) which is constantly expressed regardless of a differentiation state of cells as a control.

1. Probe

The following probes were used.

PGC-1α MB: a probe in which a 5′ end of SEQ ID NO: 1 is modified with TYE563, and a 3′ end thereof is modified with IBRQ (lowa black RQ). SEQ ID NO: 1 is a molecular beacon in which positions 1 to 7 and positions 31 to 37 are sequences constituting stem regions and complementary to each other, and positions 8 to 30 are a sequence constituting a loop structure.

Pdk1 MB: a probe in which a 5′ end of SEQ ID NO: 1 is modified with AlexaFlour488, and a 3′ end thereof is modified with IBFQ (lowa black FQ). SEQ ID NO: 1 is a molecular beacon in which positions 1 to 7 and positions 31 to 37 are sequences constituting stem regions and complementary to each other, and positions 8 to 30 are a sequence constituting a loop structure.

Actb MB: a probe in which a 5′ end of SEQ ID NO: 2 is modified with TYE665, and a 3′ end thereof is modified with IBRQ (lowa black RQ). SEQ ID NO: 2 is a molecular beacon in which positions 1 to 6 and positions 24 to 30 are sequences constituting stem regions and complementary to each other, and positions 7 to 23 are a sequence constituting a loop structure.

It was confirmed in advance that fluorescence intensities from these molecular beacons emitted fluoresce only when reacting with the mRNA encoding PGC-1α, pdk1, and actb, respectively, and that the fluorescence intensities increased depending on the amounts of the mRNAs, respectively.

2. Gelatin Nanoparticles Carrying Probe

2-1. Preparation of Gelatin Nanoparticles

Gelatin (G-2613P manufactured by Nitta Gelatin Inc.) was dissolved in 24 ml of a 0.1 M phosphate buffered aqueous solution (pH 5.0) at 37° C. To this solution, an appropriate amount of ethylenediamine was added. A hydrochloric acid aqueous solution was further added thereto to adjust the pH of the solution to 5.0. An appropriate amount of 1-ethyl-3-(3-dimethylaminopropyl) carbodiimide hydrochloride was further added thereto, and the concentration of the gelatin was adjusted to 2% by mass by addition of a 0.1 M phosphate buffer aqueous solution. This solution was stirred at 37° C. for four hours to introduce ethylenediamine into a carboxyl group of the gelatin. Thereafter, the reaction product was dialyzed in redistilled water for three days to obtain cationized gelatin in a slurry state. Thereafter, acetone as a phase separation inducer was added thereto, and the mixture was mixed at 50° C. Particles precipitated in the slurry were collected and washed with pure water to obtain cationized gelatin nanoparticles. The cationized gelatin nanoparticles are referred to as cGNS.

An apparent average particle size of cGNS was determined by a dynamic light scattering method at 37° C. using DLS-7000 manufactured by Otsuka Electronics Co., Ltd., and found to be 168.0 nm. In addition, a zeta potential of cGNS was determined by an electrophoretic light scattering method using DL S-8000 manufactured by Otsuka Electronics Co., Ltd., and found to be 8.41 mV

2-2. Carrying of Molecular Beacon by Gelatin Nanoparticles

cGNS and PGC-1α MB were mixed at room temperature for 15 minutes, and then centrifuged and washed with water to obtain gelatin nanoparticles carrying the probe. The gelatin nanoparticles are referred to as cGNS (PGC-1α MB).

cGNS and Pdk1 MB were mixed at room temperature for 15 minutes, then centrifuged and washed with water to obtain gelatin nanoparticles carrying the probe. The gelatin nanoparticles are referred to as cGNS (Pdk1 MB).

cGNS and Actb MB were mixed at room temperature for 15 minutes, then centrifuged and washed with water to obtain gelatin nanoparticles carrying the probe. The gelatin nanoparticles are referred to as cGNS (Actb MB).

The amount of the probe carried by each of cGNS (PGC-1α MB), cGNS (Pdk1 MB), and cGNS (Actb MB) was determined by a conventional method. In addition, an apparent average particle size and zeta potential of each of cGNS (PGC-1α MB), cGNS (Pdk1 MB), and cGNS (Actb MB) were determined in a similar manner to cGNS. Results thereof are indicated in Table 1. Note that the numerical values indicated in Table 1 indicate a mean±standard deviation.

TABLE 1

| | Probe carry | Average | Zeta |
|---|---|---|---|
| | amount | particle | potential |
| | (pmole/μg) | size (nm) | (mV) |
| cGNS (PGC-1α MB) | 19.4 ± 0.2 | 255.5 ± 18.1 | 10.0 ± 0.41 |
| cGNS (Pdk1 MB) | 19.7 ± 0.1 | 260.3 ± 13.0 | 9.63 ± 0.52 |
| cGNS (Actb0MB) | 19.4 ± 0.1 | 252.8 ± 18.8 | 9.21 ± 0.76 |

As is clear from Table 1, no significant change was observed among the physical properties of cGNS (PGC-1α MB), the physical properties of cGNS (Pdk1 MB), and the physical properties of cGNS (Actb MB) depending on the type (sequence) of a probe to be carried.

It was confirmed in advance that these gelatin nanoparticles were taken into cells used in the following test to the same extent, and that the expression levels and staining amounts of a target mRNA and mRNAs of various marker genes were not changed by the intake of these gelatin nanoparticles. In addition, it was confirmed in advance that the introduction amount of these gelatin nanoparticles into the cells was proportional to both contact time between the gelatin nanoparticles and the cells and the concentration of the gelatin nanoparticles, and tended to increase according to the number of the cells. The following experiment was performed under a condition in which it was confirmed that viability of the cells was not significantly reduced by intake of the gelatin nanoparticles into the cells, and that fluorescence from a probe carried by the gelatin nanoparticles could be sufficiently detected.

3. Test 1: Initial Differentiation of ES Cells

3-1. Observation of Change in Expression Level of mRNA by Differentiation State of Cells (qRT-PCR)

Mouse ES cells (EBS, 2×10⁵ cells/well) were seeded in a 6-well plate, and cultured for 48 hours in the presence of leukemia inhibitory factor (LIF) added in order to maintain an undifferentiation state. Thereafter, the medium was replaced with OptiMEM, and the cells were further cultured under a condition with or without addition of LIF. The cells were collected from each of the media one day, two days, and three days after start of the culture, RNA was extracted, and cDNA was synthesized by reverse transcription. Furthermore, using qRT-PCR, undifferentiation markers Oct-3/4, Sox2, and Nanog, and initial differentiation markers Gata4, Gata6 and Sox17 (embryonic endoderm markers), T and GSC (embryonic mesoderm markers), Pax6 and Nestin (embryonic ectoderm markers), Eomes and Cdx2 (embryonic trophectoderm markers), and the mRNA encoding PGC-1α and pdk1 were amplified. By a ΔΔCt method, first, expression levels of mRNAs of these markers were standardized using Actb as an internal standard Furthermore, the expression levels of these mRNAs under a condition with addition of LIF were standardized with respect to the expression levels of these mRNAs under a condition without addition of LIF.

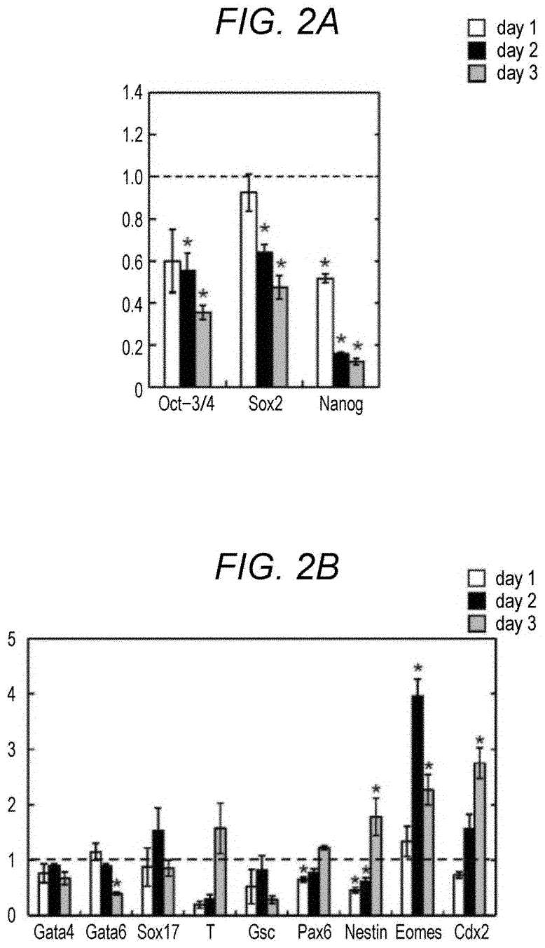

FIG. 2A is a graph illustrating an expression level of the mRNA of an undifferentiation marker, and FIG. 2B is a graph illustrating an expression level of the mRNA of an initial differentiation marker.

As is clear from FIGS. 2A and 2B, when differentiation of cells was induced under a condition without addition of LIF, the expression level of the undifferentiation marker significantly decreased over time, and the expression level of the initial differentiation marker significantly increased.

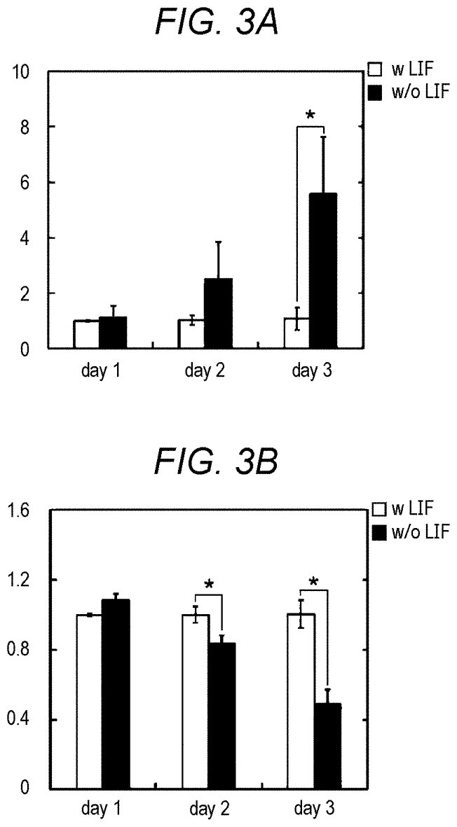

FIG. 3A is a graph illustrating an expression level of the mRNA encoding PGC-1α in a medium with addition of LIF (w LIF) or without addition of LIF (wo LIF), and FIG. 3B is a graph illustrating an expression level of pdk1 in a medium with addition of LIF (w LIF) or without addition of LIF (wo LIF).

As is clear from FIG. 3A, when differentiation of cells was induced under a condition without addition of LIF, the expression level of the mRNA encoding PGC-1α significantly increased. In addition, as is clear from FIG. 3B, when differentiation of cells was induced under a condition without addition of LIF, the expression level of pdk1 significantly decreased.

3-2. Observation of Change in Expression Level of mRNA by Differentiation State of Cells (Gelatin Nanoparticles)

Mouse ES cells (EBS, 2×10⁵ cells/well) were seeded in a 6-well plate, and cultured for 48 hours in the presence of leukemia inhibitory factor (LIF) added in order to maintain an undifferentiation state. Thereafter, the medium was replaced with OptiMEM, and the cells were further cultured under a condition with or without addition of LIF. One day, two days, and three days after start of the culture, 10 μg/mL of cGNS (PGC-1α MB) was added, and the mixture was co-cultured for one hour and then observed with a fluorescence microscope.

Similarly, one day, two days, and three days after start of the culture, 10 μg/mL of cGNS (Pdk1 MB) was added under a condition with or without addition of LIF at the time when the medium was replaced with OptiMEM, and the mixture was co-cultured for one hour and then observed with a fluorescence microscope.

Similarly, one day, two days, and three days after start of the culture, 10 μg/mL of cGNS (Actb MB) was added under a condition with or without addition of LIF at the time when the medium was replaced with OptiMEM, and the mixture was co-cultured for one hour and then observed with a fluorescence microscope

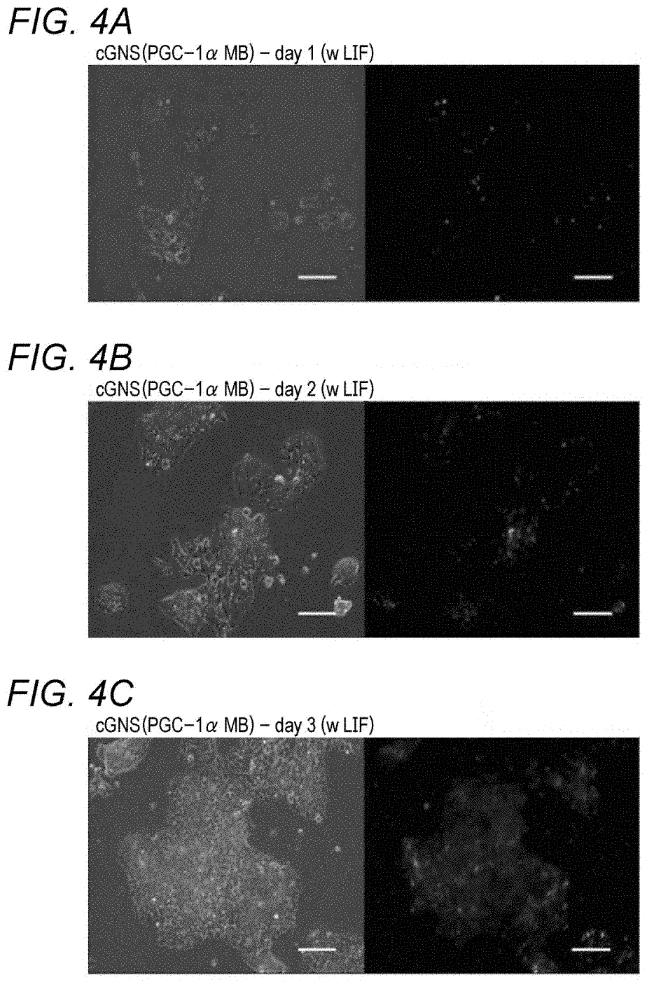

FIG. 4A illustrates a fluorescence image (right side) of a medium one day after addition of cGNS (PGC-1α MB) under a condition with addition of LIF, and an image (left side) obtained by superimposing a bright field image and the fluorescence image FIG. 4B illustrates a fluorescence image (right side) of a medium two days after addition of cGNS (PGC-1α MB) under a condition with addition of LIF, and an image (left side) obtained by superimposing a bright field image and the fluorescence image. FIG. 4C illustrates a fluorescence image (right side) of a medium three days after addition of cGNS (PGC-1α MB) under a condition with addition of LIF, and an image (left side) obtained by superimposing a bright field image and the fluorescence image.

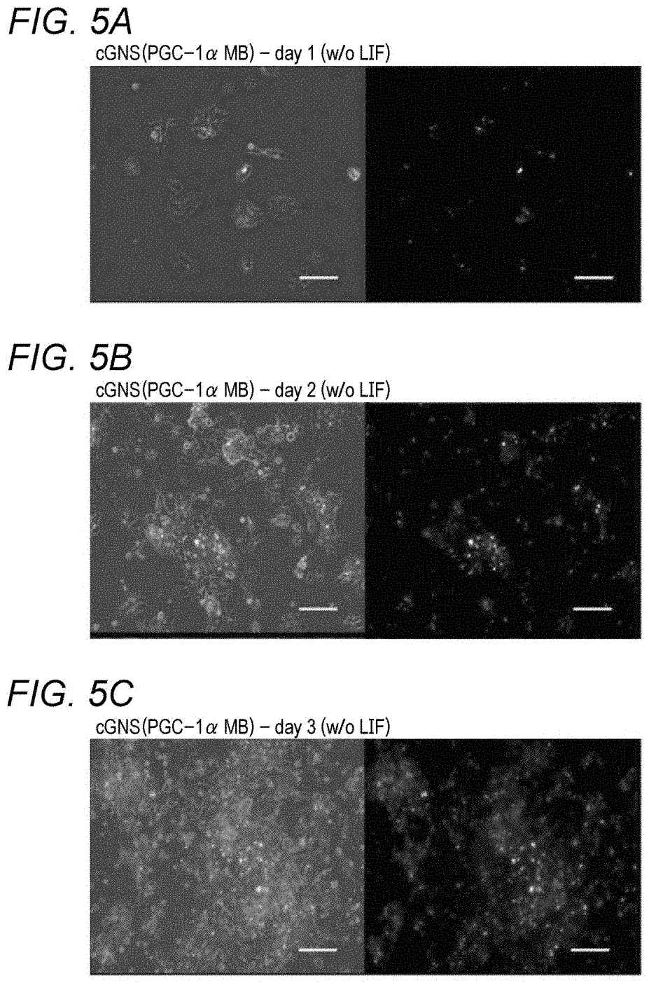

FIG. 5A illustrates a fluorescence image (right side) of a medium one day after addition of cGNS (PGC-1α MB) under a condition without addition of LIF, and an image (left side) obtained by superimposing a bright field image and the fluorescence image. FIG. 5B illustrates a fluorescence image (right side) of a medium two days after addition of cGNS (PGC-1α MB) under a condition without addition of LIF, and an image (left side) obtained by superimposing a bright field image and the fluorescence image. FIG. 5C illustrates a fluorescence image (right side) of a medium three days after addition of cGNS (PGC-1α MB) under a condition without addition of LIF, and an image (left side) obtained by superimposing a bright field image and the fluorescence image

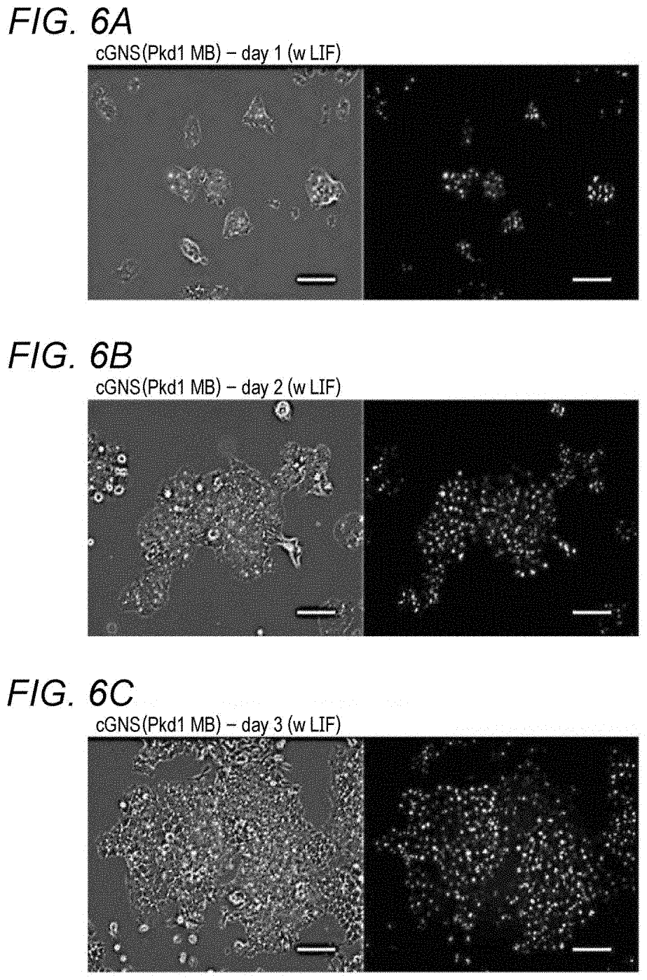

FIG. 6A illustrates a fluorescence image (right side) of a medium one day after addition of cGNS (Pdk1 MB) under a condition with addition of LIF, and an image (left side) obtained by superimposing a bright field image and the fluorescence image FIG. 6B illustrates a fluorescence image (right side) of a medium two days after addition of cGNS (Pdk1 MB) under a condition with addition of LIF, and an image (left side) obtained by superimposing a bright field image and the fluorescence image. FIG. 6C illustrates a fluorescence image (right side) of a medium three days after addition of cGNS (Pdk1 MB) under a condition with addition of LIF, and an image (left side) obtained by superimposing a bright field image and the fluorescence image

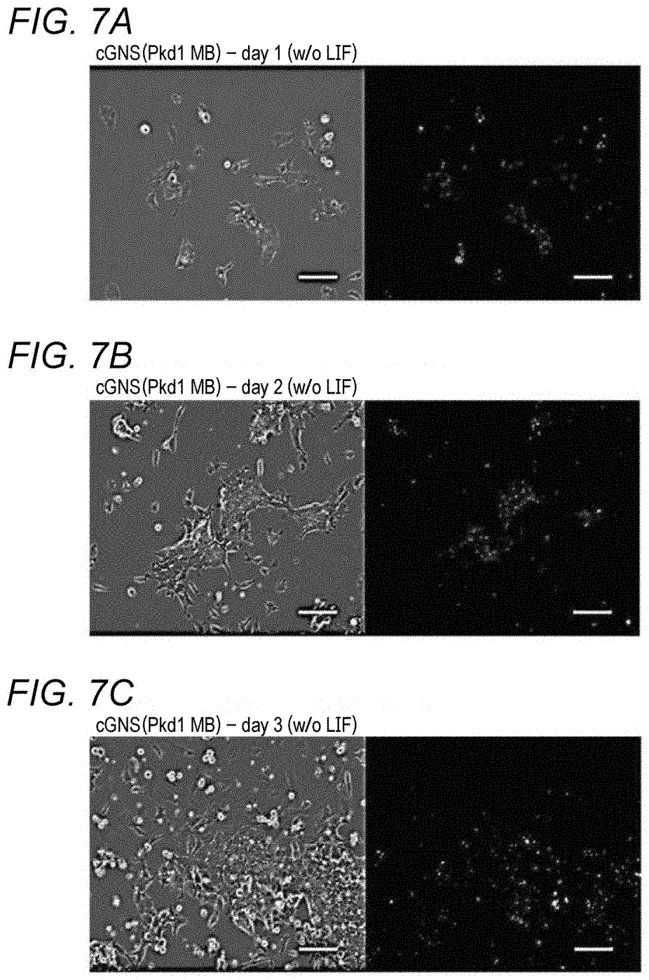

FIG. 7A illustrates a fluorescence image (right side) of a medium one day after addition of cGNS (Pdk1 MB) under a condition without addition of LIF, and an image (left side) obtained by superimposing a bright field image and the fluorescence image. FIG. 7B illustrates a fluorescence image (right side) of a medium two days after addition of cGNS (Pdk1 MB) under a condition without addition of LIF, and an image (left side) obtained by superimposing a bright field image and the fluorescence image. FIG. 7C illustrates a fluorescence image (right side) of a medium three days after addition of cGNS (Pdk1 MB) under a condition without addition of LIF, and an image (left side) obtained by superimposing a bright field image and the fluorescence image

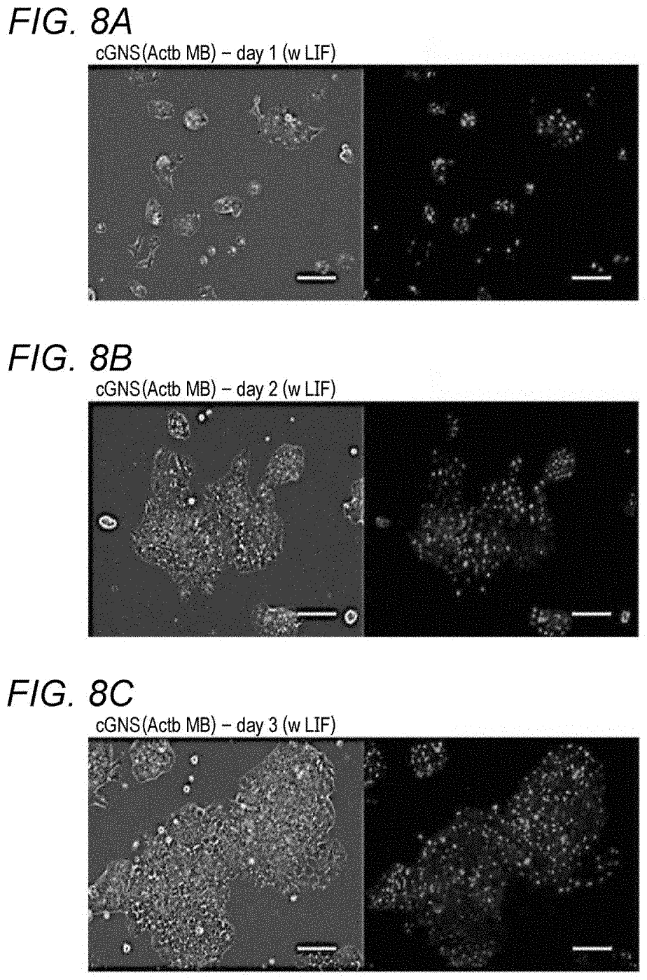

FIG. 8A illustrates a fluorescence image (right side) of a medium one day after addition of cGNS (Actb MB) under a condition with addition of LIF, and an image (left side) obtained by superimposing a bright field image and the fluorescence image FIG. 8B illustrates a fluorescence image (right side) of a medium two days after addition of cGNS (Actb MB) under a condition with addition of LIF, and an image (left side) obtained by superimposing a bright field image and the fluorescence image. FIG. 8C illustrates a fluorescence image (right side) of a medium three days after addition of cGNS (Actb MB) under a condition with addition of LIF, and an image (left side) obtained by superimposing a bright field image and the fluorescence image.

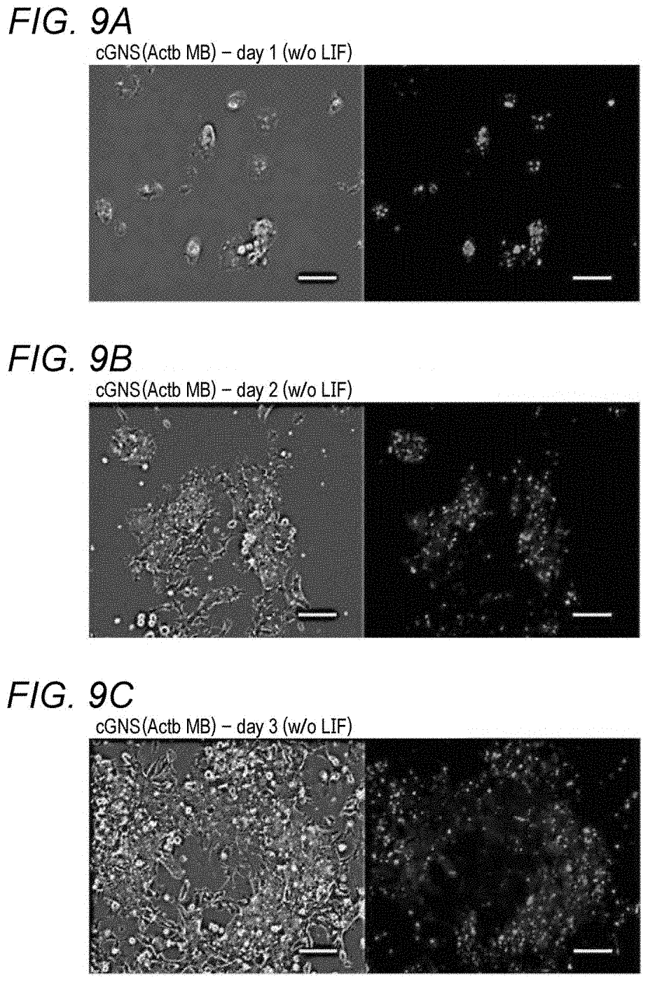

FIG. 9A illustrates a fluorescence image (right side) of a medium one day after addition of cGNS (Actb MB) under a condition without addition of LIF, and an image (left side) obtained by superimposing a bright field image and the fluorescence image. FIG. 9B illustrates a fluorescence image (right side) of a medium two days after addition of cGNS (Actb MB) under a condition without addition of LIF, and an image (left side) obtained by superimposing a bright field image and the fluorescence image. FIG. 9C illustrates a fluorescence image (right side) of a medium three days after addition of cGNS (Actb MB) under a condition without addition of LIF, and an image (left side) obtained by superimposing a bright field image and the fluorescence image

As is clear from FIGS. 4A to 4C and 5A to 5C, when LIF was added and cells were maintained in an undifferentiation state, fluorescence derived from PGC-1α MB did not increase so much in almost all cells, but when differentiation of cells was induced without addition of LIF, the fluorescence derived from PGC-1α MB remarkably increased.

As is clear from FIGS. 6A to 6C and 7A to 7C, when LIF was added and cells were maintained in an undifferentiation state, fluorescence derived from Pdk1 MB increased in almost all cells, but when differentiation of cells was induced without addition of LIF, the number of cells in which the fluorescence derived from Pdk1 MB was quenched increased.

On the other hand, as is clear from FIGS. 8A to 8C and 9A to 9C, there was no change in expression of fluorescence derived from Actb MB due to a change in the differentiation state of cells with or without addition of LIF.

Luminances of six fields randomly selected from each of the media among the fluorescence images captured by the fluorescence microscope were measured, and an average value of these luminances was taken as a fluorescence intensity of the fluorescence images.

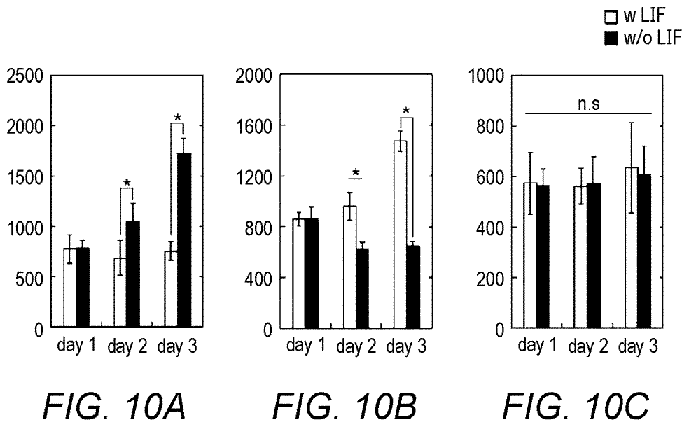

FIG. 10A is a graph illustrating a fluorescence intensity of a medium to which cGNS (PGC-1α MB) is added, FIG. 10B is a graph illustrating a fluorescence intensity of a medium to which cGNS (Pdk1 MB) is added, and FIG. 10C is a graph illustrating a fluorescence intensity of a medium to which cGNS (Actb MB) is added.

As is clear from FIG. 10A, in the fluorescence intensity when cGNS (PGC-1α MB) was introduced, the intensity from cells to which differentiation was induced without addition of LIF became larger over time than the intensity from cells in which an undifferentiation state was maintained with addition of LIF. As is clear from FIG. 10B, in the fluorescence intensity when cGNS (Pdk1 MB) was introduced, the intensity from cells to which differentiation was induced without addition of LIF became smaller over time than the intensity from cells in which an undifferentiation state was maintained with addition of LIF. Meanwhile, as is clear from FIG. 10C, in the fluorescence intensity when cGNS (Actb MB) was introduced, there was no difference between the intensity from cells to which differentiation was induced without addition of LIF and the intensity from cells in which an undifferentiation state was maintained with addition of LIF.

From these results, it is found that in the ES cells, the expression levels of the mRNA encoding PGC-1α and Pdk1 change depending on a differentiation state of the cells. Therefore, it is also found that the differentiation state of the ES cells can be determined by observing the expression level of the mRNA encoding PGC-1α or Pdk1.

Note that when the intake amount of the gelatin particles into cells was measured from radioactivity of 1251, there was no change in the intake amount into cells among cGNS (PGC-1α MB), cGNS (Pdk1 MB), and cGNS (Actb MB).

3-3. Assessment of Difference in Signal Sensitivity by Probe Introduction Method

Mouse ES cells (EBS, 2×10⁵ cells/well) were seeded in a 6-well plate, and cultured for 48 hours in the presence of leukemia inhibitory factor (LIF) added in order to maintain an undifferentiation state. Thereafter, the medium was replaced with OptiMEM, cGNS (Pdk1 MB) was added thereto, and the mixture was co-cultured for one hour. In addition, instead of cGNS (Pdk1 MB), a complex of Lipofectamine 2000, which is a gene transfer reagent containing a cationic lipid (liposome), and Pdk1 MB or Pdk1 MB alone was added, and the mixture was co-cultured for one hour similarly. Thereafter, the cells were washed with PBS, further cultured for six hours, and then observed with a fluorescence microscope.

Similarly, at the time when the medium was replaced with OptiMEM, cGNS (Actb MB), a complex of Lipofectamine 2000 and Actb MB, or Actb MB alone was added, and the mixture was co-cultured for one hour. Thereafter, the cells were washed with PBS, further cultured for six hours, and then observed with a fluorescence microscope.

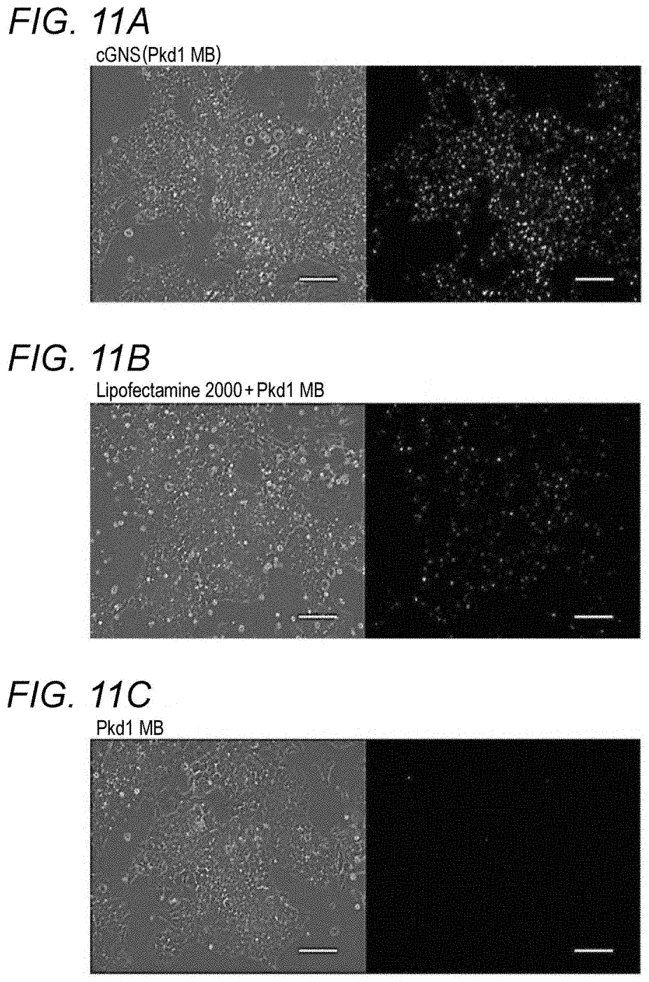

FIG. 11A illustrates a fluorescence image (right side) of a medium to which cGNS (Pdk1 MB) is added, and an image (left side) obtained by superimposing a bright field image and the fluorescence image FIG. 11B illustrates a fluorescence image (right side) of a medium to which a complex of Lipofectamine 2000 and Pdk1 MB is added, and an image (left side) obtained by superimposing a bright field image and the fluorescence image. FIG. 11C illustrates a fluorescence image (right side) of a medium to which Pdk1 MB alone is added, and an image (left side) obtained by superimposing a bright field image and the fluorescence image.

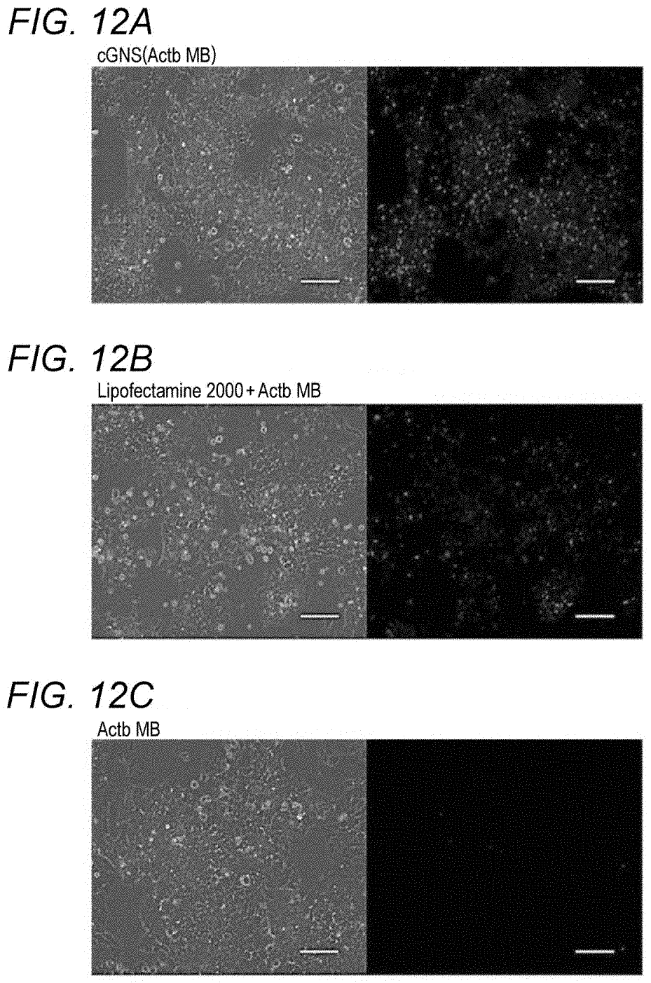

FIG. 12A illustrates a fluorescence image (right side) of a medium to which cGNS (Actb MB) is added, and an image (left side) obtained by superimposing a bright field image and the fluorescence image FIG. 12B illustrates a fluorescence image (right side) of a medium to which a complex of Lipofectamine 2000 and Actb MB is added, and an image (left side) obtained by superimposing a bright field image and the fluorescence image FIG. 12C illustrates a fluorescence image (right side) of a medium to which Actb MB alone is added, and an image (left side) obtained by superimposing a bright field image and the fluorescence image

As is clear from FIGS. 11A to 11C and 12A to 12C, when the gelatin nanoparticles carried the probe, a signal (fluorescence) was observed more strongly than when the gene introduction reagent (Lipofectamine 2000) was used or when the probe alone was added.

Not that many dead cells were observed when the gene introduction reagent (Lipofectamine 2000) was used, but almost no dead cells were observed when the gelatin nanoparticles were used.

From these results, it is found that when the gelatin nanoparticles carried the probe, a larger amount of the probe can be more safely introduced into cells.

4. Test 2: Differentiation into Nerve Cells

4-1. Observation of Change in Expression Level of mRNA by Differentiation State of Cells (qRT-PCR)

Mouse ES cells (EB5, 2×10⁵ cells/well) were seeded in a 6-well plate, and cultured for 48 hours in the presence of leukemia inhibitory factor (LIF) added in order to maintain an undifferentiation state. Thereafter, the culture medium was replaced with a neural differentiation medium (NDiff 227), and the cells were further cultured under a condition with or without addition of LIF. The cells were collected from each of the media four days, seven days, and nine days after start of the culture, RNA was extracted, and cDNA was synthesized by reverse transcription. Furthermore, using qRT-PCR, the mRNA encoding PGC-1α, pdk1, undifferentiation markers Oct-¾, Sox2, and Nanog, neural precursor cell markers Pax6 and Nestin, and a neuron marker Tubb III were amplified. By a ΔΔCt method, first, expression levels of mRNAs of these markers were standardized using Actb as an internal standard. Furthermore, the expression levels of these mRNAs under a condition with addition of LIF were standardized with respect to the expression levels of these mRNAs under a condition without addition of LIF.

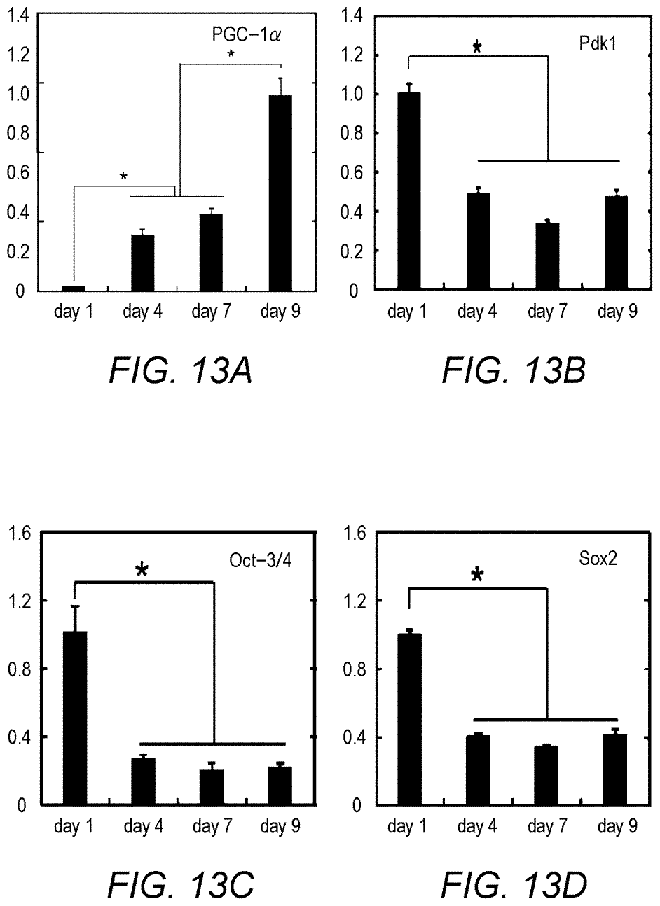

FIG. 13A is a graph illustrating an expression level of an mRNA encoding PGC-1α, FIG. 13B is a graph illustrating an expression level of the mRNA of Pdk1, FIG. 13C is a graph illustrating an expression level of the mRNA of Oct-¾, and FIG. 13D is a graph illustrating an expression level of the mRNA of Sox2.

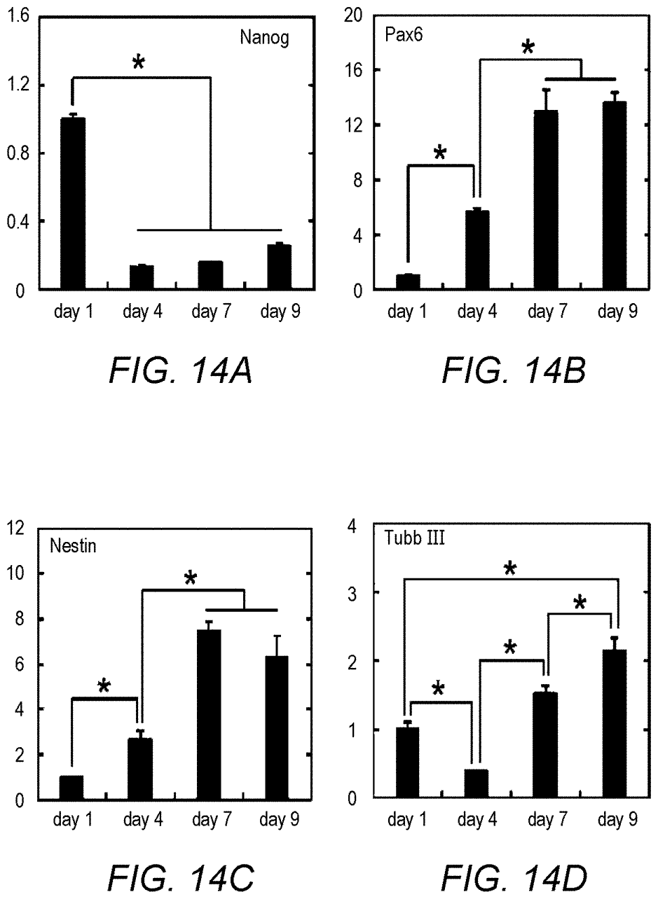

FIG. 14A is a graph illustrating an expression level of the mRNA of Nanog, FIG. 14B is a graph illustrating an expression level of the mRNA of Pax6, FIG. 14C is a graph illustrating an expression level of the mRNA of Nestin, and FIG. 14D is a graph illustrating an expression level of the mRNA of Tubb III.

As is clear from FIGS. 13A to 13D and 14A to 14D, when differentiation into nerve cells was induced, the expression level of the undifferentiation marker significantly decreased over time, the expression levels of the neural precursor differentiation marker and the neuron marker significantly increased, at the same time, the expression level of the mRNA encoding PGC-1α increased over time, and the expression level of Pdk1 decreased over time.

From these results, it is found that in the nerve cells, the expression levels of the mRNA encoding PGC-1α and Pdk1 change depending on differentiation of the cells.

4-2. Observation of Change in Expression Level of mRNA by Differentiation State of Cells (Gelatin Nanoparticles)

Mouse ES cells (EB5, 2×10⁵ cells/well) were seeded in a 6-well plate, and cultured for 48 hours in the presence of leukemia inhibitory factor (LIF) added in order to maintain an undifferentiation state. Thereafter, the culture medium was replaced with a neural differentiation medium (NDiff 227), and the cells were further cultured under a condition with or without addition of LIF. The cells were further cultured under a condition with or without addition of LIF. Four days, seven days, and nine days after start of the culture, 10 μg/mL of cGNS (PGC-1α MB) was added, and the mixture was co-cultured for one hour and then observed with a fluorescence microscope.

Similarly, four days, seven days, and nine days after start of the culture, 10 μg/mL of cGNS (Actb MB) was added under a condition with or without addition of LIF at the time when the medium was replaced with a neural differentiation medium (NDiff227), and the mixture was co-cultured for one hour and then observed with a fluorescence microscope.

Similarly, four days, seven days, and nine days after start of the culture, 10 μg/mL of cGNS (Actb MB) was added under a condition with or without addition of LIF at the time when the medium was replaced with a neural differentiation medium (NDiff227), and the mixture was co-cultured for one hour and then observed with a fluorescence microscope.

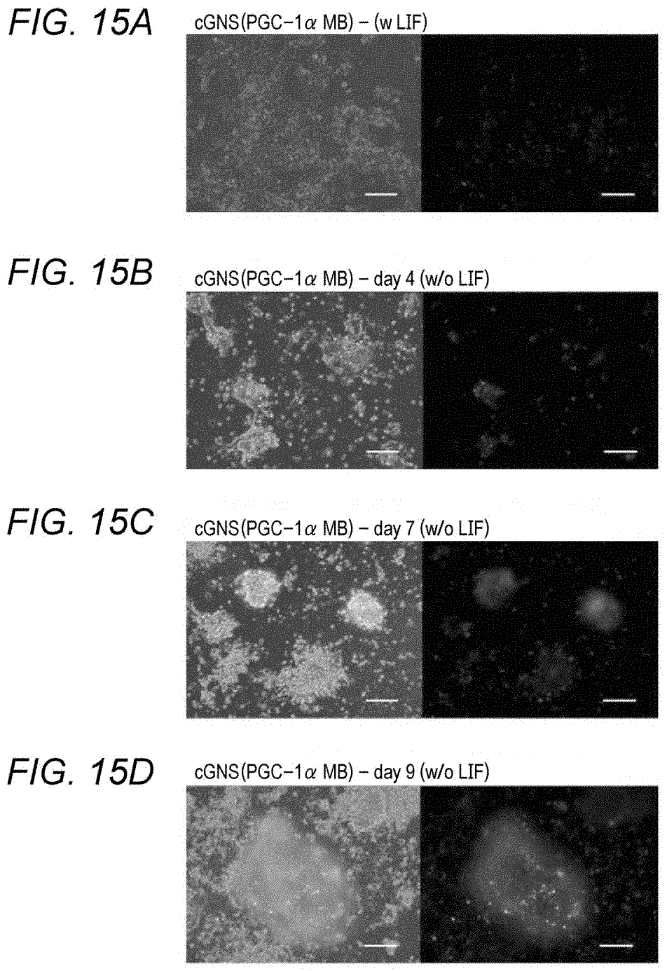

FIG. 15A illustrates a fluorescence image (right side) of a medium to which cGNS (PGC-1α MB) is added under a condition with addition of LIF, and an image (left side) obtained by superimposing a bright field image and the fluorescence image. FIG. 15B illustrates a fluorescence image (right side) of a medium four days after addition of cGNS (PGC-1α MB) under a condition without addition of LIF, and an image (left side) obtained by superimposing a bright field image and the fluorescence image FIG. 15C illustrates a fluorescence image (right side) of a medium seven days after addition of cGNS (PGC-1α MB) under a condition without addition of LIF, and an image (left side) obtained by superimposing a bright field image and the fluorescence image. FIG. 15D illustrates a fluorescence image (right side) of a medium nine days after addition of cGNS (PGC-1α MB) under a condition without addition of LIF, and an image (left side) obtained by superimposing a bright field image and the fluorescence image.

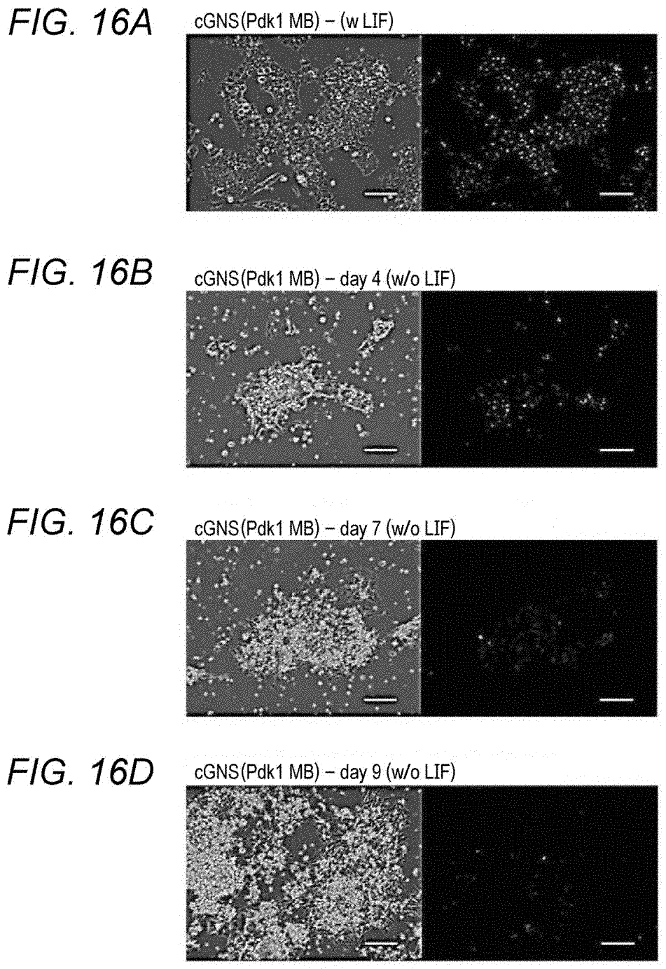

FIG. 16A illustrates a fluorescence image (right side) of a medium to which cGNS (Pdk1 MB) is added under a condition with addition of LIF, and an image (left side) obtained by superimposing a bright field image and the fluorescence image FIG. 16B illustrates a fluorescence image (right side) of a medium four days after addition of cGNS (Pdk1 MB) under a condition without addition of LIF, and an image (left side) obtained by superimposing a bright field image and the fluorescence image. FIG. 16C illustrates a fluorescence image (right side) of a medium seven days after addition of cGNS (Pdk1 MB) under a condition without addition of LIF, and an image (left side) obtained by superimposing a bright field image and the fluorescence image FIG. 16D illustrates a fluorescence image (right side) of a medium nine days after addition of cGNS (Pdk1 MB) under a condition without addition of LIF, and an image (left side) obtained by superimposing a bright field image and the fluorescence image.

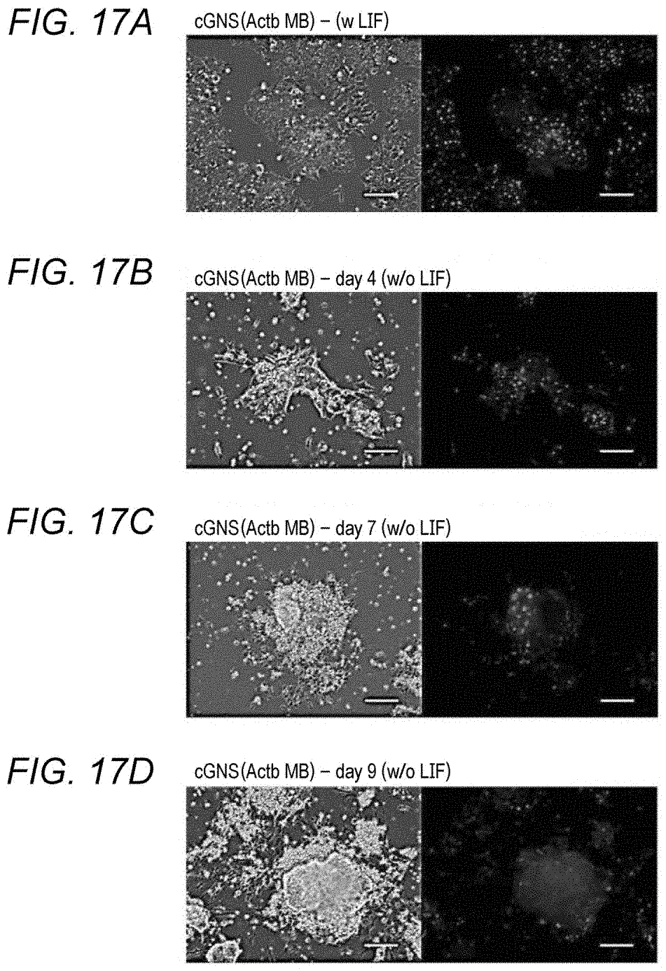

FIG. 17A illustrates a fluorescence image (right side) of a medium to which cGNS (Actb MB) is added under a condition with addition of LIF, and an image (left side) obtained by superimposing a bright field image and the fluorescence image FIG. 17B illustrates a fluorescence image (right side) of a medium four days after addition of cGNS (Actb MB) under a condition without addition of LIF, and an image (left side) obtained by superimposing a bright field image and the fluorescence image FIG. 17C illustrates a fluorescence image (right side) of a medium seven days after addition of cGNS (Actb MB) under a condition without addition of LIF, and an image (left side) obtained by superimposing a bright field image and the fluorescence image. FIG. 17D illustrates a fluorescence image (right side) of a medium nine days after addition of cGNS (Actb MB) under a condition without addition of LIF, and an image (left side) obtained by superimposing a bright field image and the fluorescence image

As is clear from FIGS. 15A to 15D, when LIF was added and cells were maintained in an undifferentiation state, fluorescence derived from PGC-1α MB did not increase so much in almost all cells, but when differentiation of cells was induced without addition of LIF, the number of cells in which the fluorescence derived from PGC-1α MB increased remarkably increased.

As is clear from FIGS. 16A to 16D, when LIF was added and cells were maintained in an undifferentiation state, fluorescence derived from Pdk1 MB was expressed in almost all cells, but when differentiation of cells was induced without addition of LIF, the number of cells in which the fluorescence derived from Pdk1 MB was quenched increased over time.

On the other hand, as is clear from FIGS. 17A to 17D, there was no change in expression of fluorescence derived from Actb MB due to a change in the differentiation state of cells with or without addition of LIF.

Luminances of six fields randomly selected from each of the media among the fluorescence images captured by the fluorescence microscope were measured, and an average value of these luminances was taken as a fluorescence intensity of the fluorescence images.

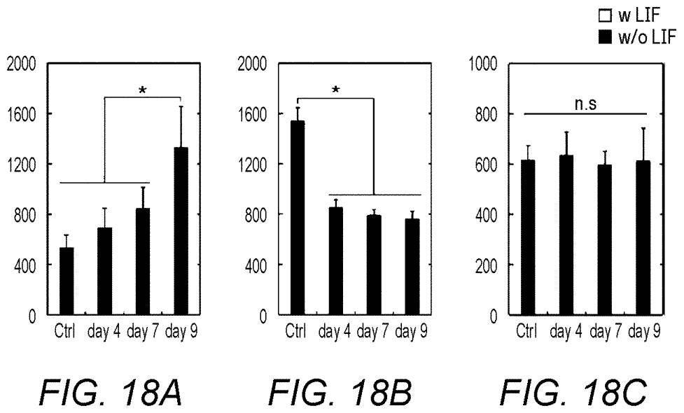

FIG. 18A is a graph illustrating a fluorescence intensity of a medium to which cGNS (PGC-1α MB) is added, FIG. 18B is a graph illustrating a fluorescence intensity of a medium to which cGNS (Pdk1 MB) is added, and FIG. 18C is a graph illustrating a fluorescence intensity of a medium to which cGNS (Actb MB) is added. Note that “Ctrl” in FIGS. 18A to 18C represents an intensity from a medium in which an undifferentiation state is maintained with addition of LIF, and “day 4”, “day 7” and “day 9” represent intensities from a medium four days, seven days, and nine days after differentiation is induced without addition of LIF, respectively.

As is clear from FIG. 18A, in the fluorescence intensity when cGNS (PGC-1α MB) was introduced, the intensity from the cells to which differentiation was induced without addition of LIF became larger over time than the intensity from the cells in which an undifferentiation state was maintained with addition of LIF. As is clear from FIG. 18B, in the fluorescence intensity when cGNS (Pdk1 MB) was introduced, the intensity from the cells to which differentiation was induced without addition of LIF became smaller than the intensity from the cells in which an undifferentiation state was maintained with addition of LIF. Meanwhile, as is clear from FIG. 18C, in the fluorescence intensity when cGNS (Actb MB) was introduced, there was no difference between the intensity from the cells to which differentiation was induced without addition of LIF and the intensity from the cells in which an undifferentiation state was maintained with addition of LIF.

From these results, it is found that in the ES cells, the expression levels of the mRNA encoding PGC-1α and Pdk1 change depending on a differentiation state of the cells. Therefore, it is also found that the differentiation state of the ES cells can be determined by observing the expression level of the mRNA encoding PGC-1α or Pdk1.

From the above-described plurality of test results, it is found that the differentiation states of a wide variety of types of cells can be determined by observing an expression level of the mRNA encoding PGC-1α or Pdk1. From these results, it is also found that the differentiation states of a wide variety of types of cells can be determined by observing an expression level of PGC-1α or PDK1 similarly.

### INDUSTRIAL APPLICABILITY

According to the present invention, a differentiation state of cells can be more easily observed. Therefore, the present invention can be applied to a wide variety of applications including regenerative medicine, discovery of a disease, and treatment of a disease, and is expected to contribute to development of these fields.

1. A method for assessing a differentiation state of cells, the method comprising:
detecting an mRNA encoding a peroxisome proliferator-activated receptor γ coactivator-1α (PGC-1α) or the peroxisome proliferator-activated receptor γ coactivator-1α (PGC-1α) in cells.

2. The method for assessing a differentiation state of cells according to claim 1, wherein the detecting is detecting the mRNA encoding PGC-1α or PGC-1α over time.

3. The method for assessing a differentiation state of cells according to claim 1, further comprising a step of assessing a differentiation state of cells based on a detection result of the mRNA encoding PGC-1α or PGC-1α.

4. The method for assessing a differentiation state of cells according to claim 3, wherein the assessing comprises determining whether cells are in a state in which metabolism by a glycolytic system is dominant or a state in which metabolism in mitochondria is activated.

5. The method for assessing a differentiation state of cells according to claim 1, wherein the detecting is simultaneously detecting the mRNA encoding PGC-1α or PGC-1α and an mRNA (Pdk1) encoding pyruvate dehydrogenase kinase 1 or pyruvate dehydrogenase kinase 1 (PDK1).

6. The method for assessing a differentiation state of cells according to claim 1, wherein the detecting comprises:
introducing a probe capable of detecting the mRNA encoding PGC-1α or PGC-1α into the cells; and
acquiring a signal from the introduced probe.

7. The method for assessing a differentiation state of cells according to claim 6, wherein the probe has a sequence complementary to at least a part of a nucleic acid sequence of the mRNA encoding PGC-1α.

8. The method for assessing a differentiation state of cells according to claim 6, wherein the probe is a molecular beacon.

9. The method for assessing a differentiation state of cells according to claim 6, wherein the introducing the probe is bringing gelatin nanoparticles carrying the probe into contact with the cells.

10. The method for assessing a differentiation state of cells according to claim 1, wherein in the cells, a state in which metabolism by a glycolytic system is dominant and a state in which metabolism in mitochondria is activated are switched by differentiation or dedifferentiation.

11. The method for assessing a differentiation state of cells according to claim 1, wherein the cells are stem cells.

12. The method for assessing a differentiation state of cells according to claim 1, wherein the cells are immune cells.

13. The method for assessing a differentiation state of cells according to claim 1 wherein the cells are cancer cells.

14. Gelatin nanoparticles for assessing a differentiation state of cells, the gelatin nanoparticles carrying a probe capable of detecting an mRNA encoding a peroxisome proliferator-activated receptor γ coactivator-1α (PGC-1α) or the peroxisome proliferator-activated receptor γ coactivator-1α (PGC-1α).

15. The gelatin nanoparticles according to claim 14, wherein the probe has a sequence complementary to at least a part of the mRNA encoding PGC-1α.

16. The gelatin nanoparticles according to claim 14, wherein the probe is a molecular beacon.

17. A gelatin nanoparticle set for assessing a differentiation state of cells, the set comprising:
gelatin nanoparticles carrying a probe capable of detecting an mRNA encoding a peroxisome proliferator-activated receptor γ coactivator-1α (PGC-1α) or the peroxisome proliferator-activated receptor γ coactivator-1α (PGC-1α); and
gelatin nanoparticles carrying a probe capable of detecting an mRNA (Pdk1) encoding pyruvate dehydrogenase kinase 1 or pyruvate dehydrogenase kinase 1 (PDK1).

18. The method for assessing a differentiation state of cells according to claim 2, further comprising assessing a differentiation state of cells based on a detection result of the mRNA encoding PGC-1α or PGC-1α.

19. The method for assessing a differentiation state of cells according to claim 2, wherein the detecting is simultaneously detecting the mRNA encoding PGC-1α or PGC-1α and an mRNA (Pdk1) encoding pyruvate dehydrogenase kinase 1 or pyruvate dehydrogenase kinase 1 (PDK1).

20. The method for assessing a differentiation state of cells according to claim 2, wherein the detecting comprises:
introducing a probe capable of detecting the mRNA encoding PGC-1α or PGC-1α into the cells; and
acquiring a signal from the introduced probe.
